(19) United States
(12) Patent Application Publication
(10) Pub. No.: US 2025/0288393 A1
(43) Pub. Date: Sep. 18, 2025

(54) METHODS AND SYSTEMS FOR OPTIMIZATION OF ORTHODONTIC TREATMENT USING PASSIVE ALIGNERS

(71) Applicant: Align Technology, Inc., San Jose, CA (US)

(72) Inventors: Anton EGOROV, Moscow (RU); Alexey SHATUNOV, Moscow (RU); Artem NOSENKO, Moscow (RU); Dmitriy Nikolaevich PAVLOV, Moscow (RU); Maria VAKHNOVETSKAYA, Moscow (RU); Igor KANEL, Cary, NC (US); Rostislav KORYAKOV, Moscow (RU); Sergey PROKOPETS, Moscow (RU); Egor SEDOV, Moscow (RU); Nilesh KONTAMWAR, Pune Maharashtra (IN); Evgenii Vladimirovich KARNYGIN, Moscow (RU)

(21) Appl. No.: 19/078,217

(22) Filed: Mar. 12, 2025

Related U.S. Application Data
(60) Provisional application No. 63/564,478, filed on Mar. 12, 2024.

(51) Int. Cl. A61C 7/00; A61C 13/34; G16H 20/40

(52) U.S. Cl. CPC A61C 7/002; A61C 13/34; G16H 20/40

(57) ABSTRACT

Methods and apparatuses for integrating passive aligners into orthodontic treatment plans. The passive aligners may be worn by a patient during treatment without applying substantial orthodontic forces on the patient's teeth. Integrating the passive aligners into the treatment plan can include separating parameter settings related to passive aligners into separate files that have different updating privileges, thereby providing a pathway to changing certain settings related to passive aligners without waiting for a full software update release cycle. A prescription service module may interface with treatment planning software to update the settings associated with passive aligners. This configuration allows for quicker inclusion of passive aligners into a treatment plan after request from a doctor. In some examples, a trained neural network model is used to interpret a request from a doctor and generate instructions to include one or more passive aligners into the orthodontic treatment plan.

### CLAIM OF PRIORITY

This patent application claims priority to U.S. Provisional Patent Application No. 63/564,478, titled “METHODS AND SYSTEMS FOR OPTIMIZATION OF ORTHODONTIC TREATMENT USING PASSIVE ALIGNERS,” filed on Mar. 12, 2024, and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Patient-removable appliances (e.g., “aligners”) have been found to be useful in orthodontic treatment in patients, and in particular for moving a patient's teeth to improve function and/or aesthetics. Treatment planning is typically performed in conjunction with the dental professional (e.g., dentist, orthodontist, dental technician, etc.) to generate a model of the patient's teeth in a final configuration, and partitioning a treatment plan into a sequence of stages (steps) corresponding to individual appliances that are worn sequentially by the patient. This process may be interactive, adjusting the staging and in some cases the final target position, based on constraints on the movement of the teeth and the dental professional's preferences. Once a final treatment plan is finalized, a series of aligners may be manufactured to implement the various stages of the treatment plan. In some cases, the aligners may be fabricated using an additive manufacturing process (e.g., three-dimensional (3D) printing) and/or molding process.

A patient may experience some discomfort wearing aligners because moving the teeth may involve the aligners applying pressure to the teeth, which in turn may apply pressure to other connected structures in the patient's mouth and jaw. Certain stages, such as initial stages, may be particularly uncomfortable as the patient may experience pressure from the aligner that they are not accustomed to. For example, the patient may be uncomfortable when wearing the first aligner for the first stage of treatment as the patient may not be used to wearing any appliance. As another example, wearing an appliance on one arch to move teeth of that arch, while the teeth of the other arch do not have an appliance because those teeth do not move, may be uncomfortable.

Although a primer appliance may be used at the beginning of primary treatment and a retainer used after treatment, such appliances may not be easily integrated into the treatment planning workflow or may otherwise not provide enough of a transition period for the patient. For example, such appliances may be made of different material from aligners, requiring different fabrication workflow. In addition, such appliances may not be as customizable as needed, for instance with respect to placement within the treatment plan as well as allowing for attachments and other aligner features and auxiliary components. Passive aligners, which are designed to keep the teeth of a dental arch in place without moving the teeth, may be integrated into the treatment plan.

### SUMMARY OF THE DISCLOSURE

As described in greater detail below, the present disclosure describes various systems and methods related to orthodontic treatment planning and fabrication of dental appliances. The systems and methods described herein may be used to integrate passive aligners into an orthodontic treatment plan. Passive aligners, also referred to as transition aligners or conversion aligners, are designed to keep the teeth of a dental arch in place without significantly moving the teeth. Including passive aligners in a treatment plan may help case patient discomfort, especially during certain stages of the treatment plan. The methods and systems are configured to consider different factors related to passive aligners in order to optimize integration of the passive aligners into the orthodontic treatment.

The methods and systems described herein are an improvement on previous treatment planning methods and systems by providing a way to change the passive aligner settings (e.g., as requested by a doctor) without depending on relatively long release cycles of treatment planning software updates. In addition, the methods and systems provide a way to limit the number of treatment stages that use passive aligners, and provides the ability to configure the maximum number of allowable passive aligners depending on criteria such as product type and/or doctor identification.

In some examples, a trained neural network model is used to interpret requests (e.g., from a doctor) for including passive aligner into a treatment plan and to generate computer readable instructions to include one or more passive aligners into the treatment plan.

According to one example, a computer-implemented method of generating an orthodontic treatment plan includes: receiving a request to include passive aligners in the orthodontic treatment plan; extracting settings related to passive aligners from treatment planning software code of a treatment planning software module; creating a treatment settings file and a product settings file and allocating the extracted settings between the treatment settings file and the product settings file, the treatment settings file including settings specific to the orthodontic treatment plan, and the product settings file including settings related to a particular product, wherein the treatment settings file and the product settings file have different updating privileges; updating the product settings file based on passive aligner settings received from a product settings administrator, wherein the product settings file is updated to indicate inclusion of passive aligners and a maximum number of allowed passive aligners; sending the updated product settings file to the treatment planning software module, wherein the treatment planning software module updates the treatment planning software code to include the passive aligners and the maximum number of allowed passive aligners according to the updated product settings file; and generating the orthodontic treatment plan via the treatment planning software module, wherein the orthodontic treatment plan includes one or more stages that includes passive aligners, and wherein a number of the one or more stages is not greater than the maximum number of allowed passive aligners. The settings related to passive aligners may be extracted from the treatment planning software code of the treatment planning software module by a prescription service module. The prescription service module may be a cloud-based service module. The prescription service module may be separate from the treatment planning software module. The settings of the treatment settings file may be unchangeable via the prescription service module. The prescription service module may save the updated product settings file in a cloud-based prescription storage database. Extracting the settings related to passive aligners may include extracting the settings specific to the orthodontic treatment plan via an internal preferences application programing interface (API), and extracting the settings related to the particular product via a product settings API. The method may further include opening a case file in the treatment planning software module in response to receiving the request to include the passive aligners. A CAD designer may open the case file. The request to include the passive aligners may be received from a doctor. The method may further include presenting an indication of allowance of passive aligners and the maximum number of allowed passive aligners according to the updated product settings file to a doctor via a client-based treatment planning software module. The method may further include presenting the orthodontic treatment plan to a dental practitioner via a display. The method may further include sending instructions to one or more manufacturing apparatuses to fabricate a set of aligners with one or more passive aligners based on the orthodontic treatment plan.

According to another example, a system includes: one or more processors; and one or more memory stores coupled to the one or more processors, wherein the one or more memory stores store computer-program instructions that, when executed by the one or more processors, perform a computer-implemented method including: receiving a request to include passive aligners in an orthodontic treatment plan; extracting settings related to passive aligners from treatment planning software code of a treatment planning software module; creating a treatment settings file and a product settings file and allocating the extracted settings between the treatment settings file and the product settings file, the treatment settings file including settings specific to the orthodontic treatment plan, and the product settings file including settings related to a particular product, wherein the treatment settings file and the product settings file have different updating privileges; updating the product settings file based on passive aligner settings received from a product settings administrator, wherein the product settings file is updated to indicate inclusion of passive aligners and a maximum number of allowed passive aligners; sending the updated product settings file to the treatment planning software module, wherein the treatment planning software module updates the treatment planning software code to include the passive aligners and the maximum number of allowed passive aligners according to the updated product settings file; and generating the orthodontic treatment plan via the treatment planning software module, wherein the orthodontic treatment plan includes one or more stages that includes passive aligners, and wherein a number of the one or more stages is not greater than the maximum number of allowed passive aligners. The method may further include sending instructions to one or more manufacturing apparatuses to fabricate a set of aligners with one or more passive aligners based on the orthodontic treatment plan. The settings related to the particular product may include jurisdiction, product type, and doctor identification. The settings specific to the orthodontic treatment plan may include placement configuration of the passive aligners in the orthodontic treatment plan. The settings related to passive aligners may be extracted from the treatment planning software code of the treatment planning software module by a prescription service module. The prescription service module may be a cloud-based service module. The prescription service module may be separate from the treatment planning software module. The settings of the treatment settings file may be unchangeable via the prescription service module. The prescription service module may save the updated product settings file in a cloud-based prescription storage database. The method may further include presenting the orthodontic treatment plan to a doctor via a display.

According to a further example, a non-transitory computing device readable medium has instructions stored thereon that are executable by one or more processors to cause one or more computing devices to perform a method including: receiving a request to include passive aligners in an orthodontic treatment plan; extracting settings related to passive aligners from treatment planning software code of a treatment planning software module; creating a treatment settings file and a product settings file and allocating the extracted settings between the treatment settings file and the product settings file, the treatment settings file including settings specific to the orthodontic treatment plan, and the product settings file including settings related to a particular product, wherein the treatment settings file and the product settings file have different updating privileges; updating the product settings file based on passive aligner settings received from a product settings administrator, wherein the product settings file is updated to indicate inclusion of passive aligners and a maximum number of allowed passive aligners; sending the updated product settings file to the treatment planning software module, wherein the treatment planning software module updates the treatment planning software code to include the passive aligners and the maximum number of allowed passive aligners according to the updated product settings file; and generating the orthodontic treatment plan via the treatment planning software module, wherein the orthodontic treatment plan includes one or more stages that includes passive aligners, and wherein a number of the one or more stages is not greater than the maximum number of allowed passive aligners. The settings related to passive aligners may be extracted from the treatment planning software code of the treatment planning software module by a prescription service module. The prescription service module may be a cloud-based service module. The method may further include sending instructions to one or more manufacturing apparatuses to fabricate a set of aligners with one or more passive aligners based on the orthodontic treatment plan.

According to another example, a computer-implemented method of generating an orthodontic treatment plan includes: receiving a request to include one or more passive aligners in the orthodontic treatment plan; generating passive aligner instructions using a trained neural network model that interprets the request to include the one or more passive aligners; gathering treatment planning assets for generating the orthodontic treatment plan using a treatment planning software module, wherein the treatment planning assets include the passive aligner instructions; and generating the orthodontic treatment plan using the treatment planning software module, wherein the orthodontic treatment plan includes a sequence of treatment stages, wherein one or more of the sequence of treatment stages includes the use of the requested one or more passive aligners. The passive aligner instructions may include a number and a placement of the requested passive aligners in the orthodontic treatment plan. The computer-implemented method may further include fabricating a set of aligners according to the treatment plan, wherein the set of aligners may include the requested one or more passive aligners. The request may be expressed in natural language. The trained neural network model may be trained using a backpropagation learning algorithm. The trained neural network model may be trained using a plurality of dental practitioner comments for different patient cases. The trained neural network model may assign an identifier for each word of the request. Generating the passive aligner instructions may include introducing an embedding layer that transforms words of the request to a semantic-meaning space. The passive aligner instructions may include a number and a placement of the passive aligners in the treatment plan, wherein the placement of the passive aligners may include a beginning, a middle, or an end of the treatment plan. Generating the passive aligner instructions may include converting a protocol language into a treatment settings file format that may be readable by the treatment planning software module. The trained neural network model may be a recurrent neural network model. The recurrent neural network model may be a long short-term memory (LSTM) recurrent neural network model. The treatment planning assets may be gathered from one or more computers that may be remote from a treatment planning software module. The remote computer may be a dental practitioner's computer. Generating the passive aligner instructions may include tokenizing the request. Generating the orthodontic treatment plan may include determining which one or more stages of the treatment plan include the requested one or more passive aligners. The requested one or more passive aligners may be integrated in corresponding one or more stages of the treatment plan after the stages are created. Generating the passive aligner instructions may include splitting the request into groups of words, sentences or phrases and separating the groups of words, sentences or phrases with separators. The separators may include semicolons and newline characters.

According to an additional example, a system includes: one or more processors; and one or more memory stores coupled to the one or more processors, wherein the one or more memory stores store computer-program instructions that, when executed by the one or more processors, perform a computer-implemented method including: receiving a request to include one or more passive aligners in the orthodontic treatment plan; generating passive aligner instructions using a trained neural network model that interprets the request to include the one or more passive aligners; gathering treatment planning assets for generating the orthodontic treatment plan using a treatment planning software module, wherein the treatment planning assets include the passive aligner instructions; and generating the orthodontic treatment plan using the treatment planning software module, wherein the orthodontic treatment plan may include a sequence of treatment stages, wherein one or more of the sequence of treatment stages may include the use of the requested one or more passive aligners. The system may include: a passive aligner request module that receives the request and generates the passive aligner instructions; and a treatment planning module that gathers the treatment planning assets and generates the orthodontic treatment plan. The passive aligner request module may be on a first computer that may be remote to a second computer with the treatment planning module. The passive aligner instructions may include a number and a placement of the requested passive aligners in the orthodontic treatment plan. The computer-implemented method may further include sending instructions to one or more manufacturing apparatuses to fabricate a set of aligners with one or more passive aligners based on the orthodontic treatment plan. The request may be expressed in natural language. The trained neural network model may be trained using a backpropagation learning algorithm. The trained neural network model may be trained using a plurality of dental practitioner comments for different patient cases. The trained neural network model may assign an identifier for each word of the request. Generating the passive aligner instructions may include introducing an embedding layer that transforms words of the request to a semantic-meaning space. The placement of the passive aligners may include a beginning, a middle, or an end of the treatment plan. Generating the passive aligner instructions may include converting a protocol language into a treatment settings file format that may be readable by the treatment planning software module. The trained neural network model may be a recurrent neural network model. The recurrent neural network model may be a long short-term memory (LSTM) recurrent neural network model. The treatment planning assets may be gathered from one or more computers that may be remote from the treatment planning software module. At least one of the remote one or more computers may be a dental practitioner's computer. Generating the orthodontic treatment plan may include determining which one or more stages of the treatment plan include the requested one or more passive aligners. The requested one or more passive aligners may be integrated in corresponding one or more stages of the treatment plan after the stages are created. Generating the passive aligner instructions may include splitting the request into groups of words, sentences or phrases and separating the groups of words, sentences or phrases with separators.

According to a further example, a non-transitory computing device readable medium has instructions stored thereon that are executable by one or more processors to cause one or more computing devices to perform a method including: receiving a request to include one or more passive aligners in the orthodontic treatment plan; generating passive aligner instructions using a trained neural network model that interprets the request to include the one or more passive aligners; gathering treatment planning assets for generating the orthodontic treatment plan using a treatment planning software module, wherein the treatment planning assets include the passive aligner instructions; and generating the orthodontic treatment plan using the treatment planning software module, wherein the orthodontic treatment plan may include a sequence of treatment stages, wherein one or more of the sequence of treatment stages may include the use of the requested one or more passive aligners. The passive aligner instructions may include a number and a placement of the requested passive aligners in the orthodontic treatment plan. The method may further include sending instructions to one or more manufacturing apparatuses to fabricate a set of aligners with one or more passive aligners based on the orthodontic treatment plan. The trained neural network model may be a recurrent neural network model.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a diagram illustrating an example of a computing environment.

FIG. 2A is a diagram illustrating an example of a prescription (Rx) service module.

FIG. 2B is a diagram illustrating an example file configuration for saving passive aligner settings.

FIG. 3 is a diagram illustrating an example workflow overview scheme for including passive aligners in an orthodontic treatment plan.

FIG. 4 is a diagram illustrating an example of an order opening process for including passive aligners in an orthodontic treatment plan.

FIG. 5 is a flowchart indicating an example process for generating treatment plan(s) and fabricating a set of aligners that include one or more passive aligners.

FIG. 6 is a chart illustrating example planned tooth movements for the upper and lower jaws at different stages of a treatment plan.

FIG. 7 is a diagram illustrating an example context interpretation using a long short-term memory (LSTM) recurrent neural network (RNN).

FIG. 8 is a diagram illustrating an example workflow for including a passive aligner in a treatment plan.

FIG. 9. is a diagram illustrating an example use of an automated dataset preparation script.

FIG. 10 is a diagram illustrating an example classification.

FIGS. 11A and 11B are graphs illustrating example epoch counts of an example prototype training.

FIG. 12 is a graph illustrating an example probability distribution of an example neural network output.

FIG. 13 is an example of passive aligner instruction for passive aligner placement.

FIG. 14 is a diagram illustrating an example workflow for automated passive aligner placement.

FIG. 15 is a flowchart indicating an example process for generating a treatment plan including using a neural network to interpret requests for passive aligners.

### DETAILED DESCRIPTION

The methods and apparatuses (e.g., systems, devices, etc., including software, hardware and/or firmware) described herein relate to the field of orthodontics, and more particularly to passive appliances, also referred to as transition or conversion appliances. The passive appliances may be passive aligners that are designed to keep the teeth of a dental arch in place without moving the teeth. The use of passive aligners in certain stages of a treatment plan may help ease patient discomfort.

In general, orthodontic treatment planning involves evaluating a patient's teeth in their current configuration, determining a target (e.g., final) configuration, and generating treatment plan to transition the teeth from their current configuration to the target configuration. This process involves multiple steps. For example, one or more images of the patient's teeth in their current configuration can be taken. The images are then used to generate a three-dimensional model (3D) (e.g., virtual model) of the patient's teeth. The doctor and patient decide on treatment goals for treating the patient's teeth. The 3D model is then used to generate the target (e.g., final) 3D model representing the patient's teeth in a desired configuration based on the treatment goals. Treatment planning software can be used to create a treatment plan that includes multiple intermediate stages for incrementally moving the teeth toward the target configuration. Each stage may be associated with a dental aligner so that the patient can wear a series of dental aligners to implement the treatment plan.

Some treatment plans include the use of passive aligners, which are aligners that are designed to keep the teeth of a dental arch in place without moving (or not significantly moving) the teeth. One use case of passive aligners is to keep the teeth of one arch (upper or lower) in place while teeth in the opposite arch are still moving. This may occur, for example, when treatment of the teeth of one arch is complete but teeth of the opposing arch is still required according to the treatment plan. In some cases, dual-arch passive aligners (DPAs) are used, where passive aligners (also referred to as conversion aligners) are worn on both upper and lower arches of a patient. Dual-arch passive aligners may be worn as initial, intermediate and/or final aligners in the series of aligners, for example, to improve patient comfort. Examples of passive appliances are described in International Patent Application publication WO2023115062A1, filed on Dec. 19, 2022, which is incorporated herein by reference in its entirety.

The term “passive aligner” may refer to a single passive aligner that is configured to be worn on one dental arch (upper or lower). The term “dual-passive aligner” (e.g., abbreviated as “DPA”) may refer to a pair of passive aligners that are configured to be worn on both dental arches (upper and lower) simultaneously for at least part of a particular stage of a treatment plan. The term “dual-passive aligner” (e.g., abbreviated as “DPA”) is used in several instances herein. In these cases, it should be understood that in single passive aligner (for the upper or lower jaw) may be implemented instead of or in addition to a dual passive aligner. In general, the methods described herein may be useful for configuring any settings using the product settings file (PSF) including (but not limited to) single passive aligners and dual passive aligners.

Integrating passive appliances into a treatment plan may involve collecting various types of information from different sources and integrating this information so that it can be used to optimize the treatment plan. For example, clinical information about the patient's dental condition and treatment goals may be collected from the doctor and various available and current treatment types may be extracted from the treatment planning software. A prescription service module may define limits as to whether to integrate passive aligners into a treatment plan, and if allowed, define how many passive aligners to allow based on case-specific criteria. Thus, the prescription service module provides a way to limit the number of treatment stages that use passive aligners, and provides the ability to configure the availability and maximum number of passive aligners depending on criteria, for example, based on product type and/or doctor. The prescription service module may also provide a way to change the configuration of the passive aligner settings without depending on relatively long release cycles of treatment planning software updates.

In general, these methods and apparatuses (systems, devices, etc., including software, hardware and/or firmware) may be used at one or more parts of a dental computing environment, including as part of an intraoral scanning system, doctor system, treatment planning (e.g., technician) system, patient system, and/or fabrication system. In particular, these methods and apparatuses may be used as part of a treatment planning system that integrates dual-arch passive aligners into an orthodontic treatment plan.

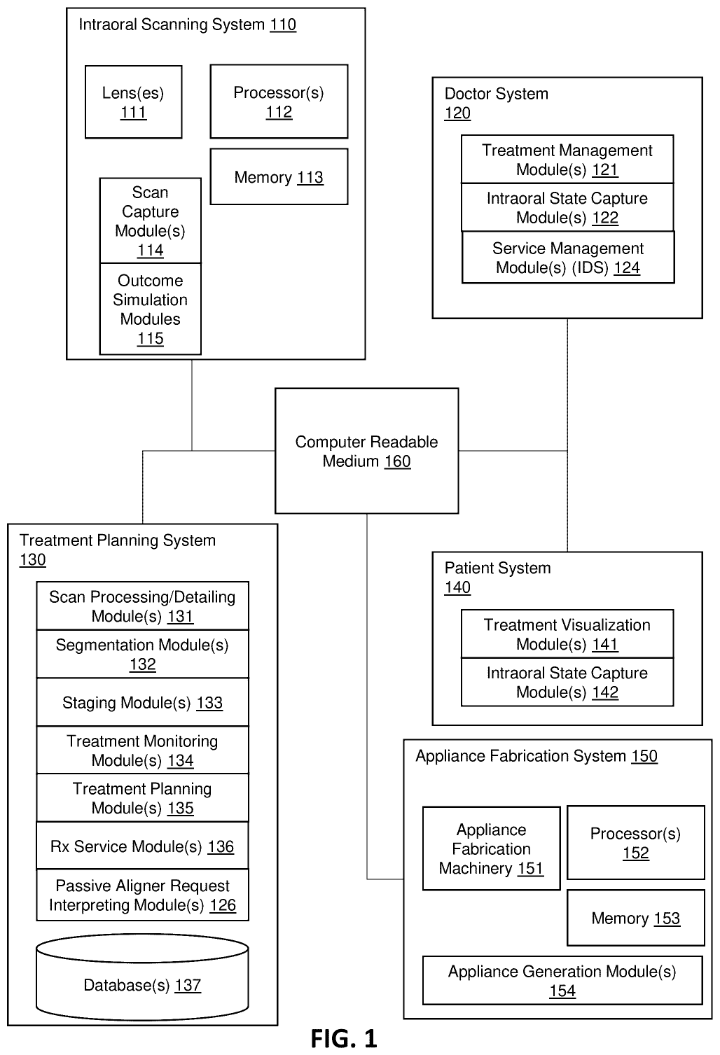

FIG. 1 is a diagram illustrating one variation of a computing environment 100 that may generate one or more orthodontic treatment plans specific to a patient, and fabricate dental appliances that may accomplish the treatment plan to treat a patient, under the direction of a dental professional. The example computing environment 100 shown in FIG. 1 includes an intraoral scanning system 110, a doctor system 120, a treatment planning system 130 (e.g., technician system), a patient system 140, an appliance fabrication system 150, and computer-readable medium 160. Each of these systems may be referred to equivalently as a sub-system of the overall system (e.g., computing environment). Although shown as discrete systems, some or all of these systems may be integrated and/or combined. In some variations a computing environment (dental computing system) 100 may include just one or a subset of these systems (which may also be referred to as sub-systems of the overall system 100). As mentioned, one or more of these systems may be combined or integrated with one or more of the other systems (sub-systems), such as, e.g., the patient system and the doctor system may be part of a remote server accessible by doctor and/or patient interfaces. In some variations each of the systems 110, 120, 140 and 160 may include one or more computers. The computers may be in the same location or in separate (e.g., remote) locations. In some examples, a first computer of the doctor system 120 is remote to a second computer of the treatment planning system 130.

The computer readable medium 160 may divided between all or some of the systems (subsystems); for example, the treatment planning system and appliance fabrication system may be part of the same sub-system and may be on a computer readable medium 160. Further, each of these systems may be further divided into sub-systems or components that may be physically distributed (e.g., between local and remote processors, etc.) or may be integrated.

An intraoral scanning system may include an intraoral scanner as well as one or more processors for processing images. For example, an intraoral scanning system 110 can include optics 111 (e.g., one or more lenses, filters, mirrors, etc.), processor(s) 112, a memory 113, scan capture module 114, and outcome simulation module 115. In general, the intraoral scanning system 110 can capture one or more images of a patient's dentition. Use of the intraoral scanning system 110 may be in a clinical setting (doctor's office or the like) or in a patient-selected setting (the patient's home, for example). In some cases, operations of the intraoral scanning system 110 may be performed by an intraoral scanner, dental camera, cell phone or any other feasible device.

The optical components 111 may include one or more lenses and optical sensors to capture reflected light, particularly from a patient's dentition. The scan capture module 114 can include instructions (such as non-transitory computer-readable instructions) that may be stored in the memory 113 and executed by the processor(s) 112 to can control the capture of any number of images of the patient's dentition.

For example, the outcome simulation module 115, which may be part of the intraoral scanning system 110, can include instructions that simulate the tooth positions based on a treatment plan. Alternatively or additionally, in some examples, the outcome simulation module 115 can import tooth number information from 3D models onto 2D images to assist in determining an outcome simulation.

Any of the component systems or sub-systems of the dental computing environment 100 may access or use the 3D model of the patient's dentition generated by the methods and apparatuses described herein. For example, the doctor system 120 may include a treatment management module (e.g., ClinCheck) 121 and an intraoral state capture module 122 that may access or use the 3D model. The doctor system 120 may provide a “doctor facing” interface to the computing environment 100. The treatment management module 121 can perform any operations that enable a doctor or other clinician to manage the treatment of any patient. In some examples, the treatment management module 121 may provide a visualization and/or simulation of the patient's dentition with respect to a treatment plan.

The intraoral state capture module 122 can provide images of the patient's dentition to a clinician through the doctor system 120. The images may be captured through the intraoral scanning system 110 and may also include images of a simulation of tooth movement based on a treatment plan.

In some examples, the treatment management module 121 can enable the doctor to modify or revise a treatment plan, particularly when images provided by the intraoral state capture module 122 indicate that the movement of the patient's teeth may not be according to the treatment plan. The doctor system 120 may include one or more processors configured to execute any feasible non-transitory computer-readable instructions to perform any feasible operations described herein.

The doctor system 120 may also include a service management module 124 (e.g., IDS) that provides a user interface for a doctor to manage treatment workflow. For example, the doctor may enter information and access information associated with each patient (e.g., clinical conditions for each patient, patient address, etc.), treatment account information, clinical preferences (e.g., preferences related to passive aligners, preferences related to interproximal reduction (IPR), etc.), access educational information (e.g., instructional slides, videos, etc.) and/or ways to order treatment products.

The doctor system 120 may further include a passive aligner request interpreting module 126 that is configured to interpret requests (e.g., from a dental practitioner) for including one or more passive aligners in the treatment plan. In some examples, the passive aligner request interpreting module 126 may interpret the requests using a neural network model. In some examples the passive aligner request interpreting module 126 is remote from the doctor system, but included, e.g., as part of the treatment planning system 130 or other sub-system, and may be accessed remotely (e.g., through a cloud-based system).

The TSF and PSF may be delivered to the case in a similar manner. For example, the TSF and PSF files may be text files, e.g., in .json format (a common standard for convenient transfer of parameters in applications) that contain a list of settings for a specific order. The TSF includes the clinical settings level, e.g., settings that are defined for the treatment as a whole. The technician typically cannot change these settings in the course of treatment (and may be referred to as ‘unchangeable’). The PSF includes the product settings level, and may refer to settings that are specific to a particular product; as described herein, the technician has the ability to change these settings (and may be referred to as ‘changeable’).

The treatment planning system 130 may include any of the methods and apparatuses described herein. The treatment planning system 130 may include scan processing/detailing module 131, segmentation module 132, staging module 133, treatment monitoring module 134, treatment planning module 135, prescription (Rx) service module 136, and databases 137. In general, the treatment planning system 130 can determine a treatment plan for any feasible patient. The scan processing/detailing module 131 can receive or obtain dental scans (such as scans from the intraoral scanning system 110) and can process the scans to “clean” them by removing scan errors and, in some cases, enhancing details of the scanned image. The treatment planning system 130 may perform segmentation. For example, a treatment planning system may include a segmentation module 132 that can segment a dental model into separate parts including separate teeth, gums, jaw bones, and the like. In some cases, the dental models may be based on scan data from the scan processing/detailing module 131.

The staging module 133 may determine different stages of a treatment plan. Each stage may correspond to a different dental aligner. The staging module 133 may also determine the final position of the patient's teeth, in accordance with a treatment plan. Thus, the staging module 133 can determine some or all of a patient's orthodontic treatment plan. In some examples, the staging module 133 can simulate movement of a patient's teeth in accordance with the different stages of the patient's treatment plan.

The treatment monitoring module 134 can monitor the progress of an orthodontic treatment plan. In some examples, the treatment monitoring module 134 can provide an analysis of progress of treatment plans to a clinician. The treatment planning module 135 can include treatment planning software code that uses various settings/parameters to generate one or more treatment plans. The treatment planning module 135 can be configured to determine one or more treatment paths for a patient's teeth from a current configuration to a target (e.g., final) configuration, and a number of intermediate configurations between the current and target configuration. The treatment planning software code and the orthodontic treatment plans may be stored in a treatment planning database (e.g., one of the databases 137). The treatment planning software may be updated according to software update release cycles.

The Rx service module 136 provides another pathway for changing passive aligner settings other than via the software update release cycles of the treatment planning module 135. The Rx service module 136 may be on a remote computer in relation to a computer with the treatment planning module 135, or may be on the same computer as the treatment planning module 135. In some examples, the Rx service module 136 is a cloud-based service module. The Rx service module 136 may be used to determine whether passive aligners are allowed (e.g., based on product type, doctor, etc.), and if allowed, the maximum number of allowed passive aligners. As described in detail later, the Rx service module 136 can store information related to passive aligner settings in a product application settings file, for example, in a prescription (Rx) storage database (e.g., one of the databases 137). The Rx service module 136 with the associated prescription storage database may be part of a cloud-based service that is stored in one or more different computers than the treatment planning module 135.

Although not shown here, the treatment planning system 130 can include one or more processors configured to execute any feasible non-transitory computer-readable instructions to perform any feasible operations described herein.

The patient system 140 can include a treatment visualization module 141 and an intraoral state capture module 142. In general, the patient system 140 can provide a “patient facing” interface to the computing environment 100. The treatment visualization module 141 can enable the patient to visualize how an orthodontic treatment plan has progressed and also visualize a predicted outcome (e.g., a final position of teeth).

In some examples, the patient system 140 can capture dentition scans for the treatment visualization module 141 through the intraoral state capture module 142. The intraoral state capture module can enable a patient to capture his or her own dentition through the intraoral scanning system 110. Although not shown here, the patient system 140 can include one or more processors configured to execute any feasible non-transitory computer-readable instructions to perform any feasible operations described herein.

The appliance fabrication system 150 can include appliance fabrication machinery 151, processor(s) 152, memory 153, and appliance generation module 154. In general, the appliance fabrication system 150 can directly or indirectly fabricate aligners to implement an orthodontic treatment plan. In some examples, the orthodontic treatment plan may be stored in one of the databases 137.

The appliance fabrication machinery 151 may include any feasible implement or apparatus that can fabricate any suitable dental aligner. The appliance generation module 154 may include any non-transitory computer-readable instructions that, when executed by the processor(s) 152, can direct the appliance fabrication machinery 151 to produce one or more dental aligners. The memory 153 may store data or instructions for use by the processor(s) 152. In some examples, the memory 153 may temporarily store a treatment plan, dental models, or intraoral scans.

The computer-readable medium 160 may include some or all of the elements described herein with respect to the computing environment 100. The computer-readable medium 160 may include non-transitory computer-readable instructions that, when executed by a processor, can provide the functionality of any device, machine, or module described herein.

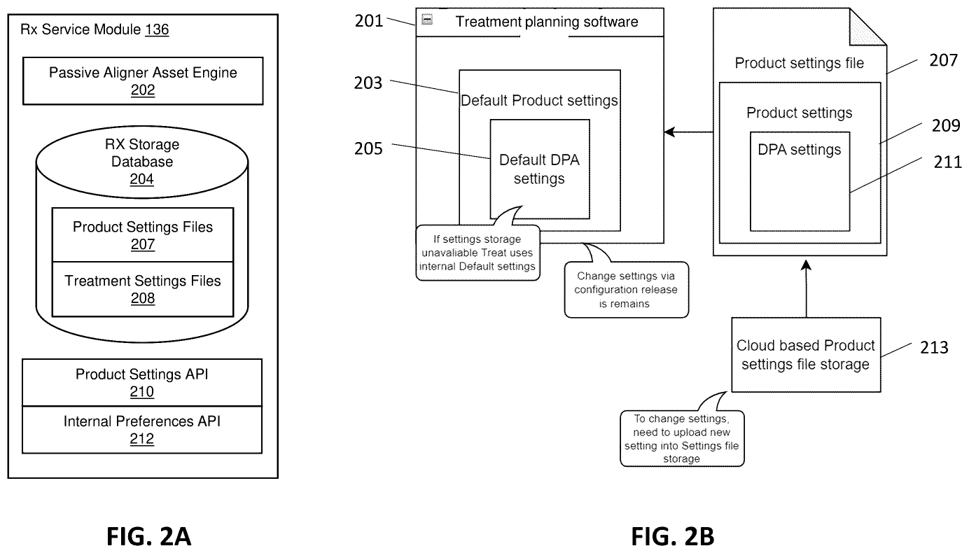

FIG. 2A shows a diagram of an example prescription (Rx) service module 136. The Rx service module 136 may be stored in a different database than the treatment planning module 135. In some examples, the Rx service module 136 may be a cloud-based module. The Rx service module 136 includes a passive aligner asset engine 202, which includes software code for determining assets related to passive aligners. The passive aligner asset engine 202 is configured to retrieve/extract settings related to passive aligners from treatment planning software code of the treatment planning module 135. A product settings API 210 is configured to retrieve/extract assets that are specific to the current product (product settings) and to create a product settings file 207. The internal preferences API 212 is configured to retrieve/extract assets related to a treatment (treatment settings) and to create a treatment settings file 208. As described herein, the product settings file 207 and the treatment settings file 208 can include different assets/parameters/values related to passive aligners. The passive aligner asset engine 202 can store the product settings file 207 and the treatment settings file 208 in a prescription (Rx) storage database.

FIG. 2B shows a diagram of an example file configuration for saving passive aligner (DPA) settings. Default passive aligner (DPA) settings 205 are saved as default product settings 203, which the treatment planning software 201 of the treatment planning module 135 uses to generate one or more treatment plans. If the default passive aligner settings 205 are unavailable, the treatment planning software 201 uses internal default settings. The default passive aligner (DPA) settings 205 (or internal default settings) may be changed via the release cycles when the treatment planning software 201 code is updated. Updating the treatment planning software 201 may involve changing any of a number of aspects of the treatment planning software 201 (e.g., besides the default passive aligner (DPA) settings 205), which may require relatively lengthy individual testing and verification processes. Therefore, updates to the treatment planning software 201 may be relatively long.

A separate product settings file 207 may be used to provide an alternative route to changing the default passive aligner (DPA) settings 205. The default passive aligner (DPA) settings 205 for treatment types may be extracted from the code of the treatment planning software 201 and allocated to separate files: a treatment setting file and a product setting file 207. As mentioned, TSF stans for “Treatment settings file”, PSF stans for “Product settings file”, both contained in In the “Latest Treatment application settings files” on this FIGS. 1 and 2A-2B. In this example RxService does not extract anything from the treatment planning settings (e.g., treatment planning code); this may be handled by the an administer (or administration team) in the process of migrating the settings to our platform. The methods described herein may look at what parameters are needed for each product and may configure RxService to generate the necessary asserts (including TSF and PSF) and send them to the order, for use in the treatment process. The RxService module may generate assets only based on its own configuration files.

The product setting file 207 contains settings that are specific to the current product and that can be changed (e.g., by a technician) during the treatment process. The treatment setting file contains settings that are specific to a treatment and are unchangeable.

The product settings file 207 may be stored in a cloud-based storage database 213. As described herein, the passive aligner (DPA) settings 211 may be modified (e.g., from default settings) and uploaded into the product settings file 207. The product settings file 207 (with the modified passive aligner (DPA) settings 211) may then replace the default product settings 203, which the treatment planning software 201 uses to generate one or more treatment plans. In cases where the product settings file 207 has unviable passive aligner (DPA) settings, the code of the treatment planning software 201 can use the internal default setting values.

In addition, the software development team can deliver changes to the product settings files 207 independently of the developers of the treatment planning software 201, which increases the speed at which passive aligner (DPA) settings 211 can be delivered to the production environment.

The product settings file 207 defines the parameters of the passive aligner (DPA) settings 211. Example parameters may include: whether a passive aligner is allowed or not allowed based on a particular case flow; whether a passive aligner is allowed or not allowed based on a particular treatment type; whether a passive aligner is allowed or not allowed based on a particular doctor; and/or if a passive aligner is allowed, how many stages of the treatment plan are allowed to include passive aligners.

The product settings file 207 may be generated by a prescription service module (or submodule). As discussed previously, the prescription service module may be a cloud-based application. The prescription service module creates the product settings file 207 based on its own product-specific configuration template for each submitted order (e.g., request for addition of passive aligner in the treatment plan). For example, the passive aligner (DPA) settings 211 may include values for various parameters such as order treatment type, geographical region, doctor, category, patient type, and/or other factors. A product configuration template may be product-specific and be deployed with a quick release cycle. This allows the software development team to set values for the passive aligner (DPA) settings 211 by changing the product configuration template of these settings in the prescription service module independent of treatment planning software 201 releases.

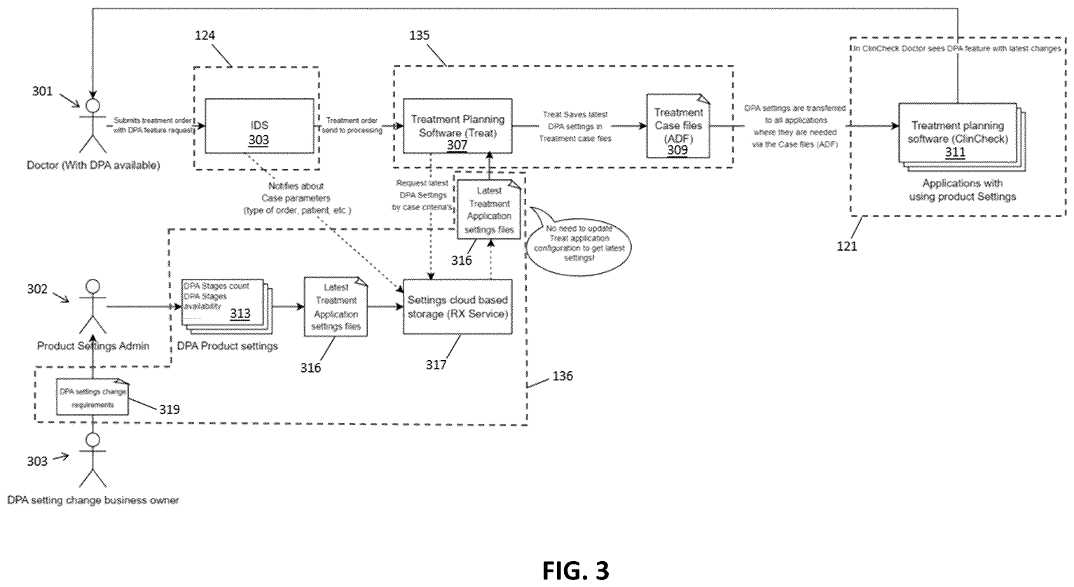

FIG. 3 shows an example workflow overview scheme. A doctor 301 who has passive aligner (DPA) privileges available submits a treatment order with passive aligner (DPA) feature request via a service management module (IDS) interface 303 of a service management module (IDS) 124. The treatment order is sent to the Treatment Planning Software 307 of a treatment planning module 135 for processing. In addition, a cloud-based prescription (Rx) service engine and storage service 317 of an Rx service module 136 is notified about the case parameters (e.g., treatment type, geographical region, doctor, category, patient type, etc.).

A passive aligner (DPA) setting change business owner 303 sends passive aligner (DPA) settings change requirements 319 to a product settings administrator 302. The product settings administrator 302 uses the requirements 319 to determine the passive aligner (DPA) product settings 313, which includes the maximum number of passive aligner (DPA) stages (e.g., based on the availability of passive aligner stages). The passive aligner (DPA) product settings 313 are used to create treatment settings files 316, which include a product settings treatment file 207 and a treatment settings file 208. The treatment settings files 316 include assets with regard to passive aligners, such as whether passive aligners (DPA) are allowed, and if allowed, the maximum number of allowed passive aligners (DPA). The prescription service (RX Service) module 317 also requests/extracts the latest passive aligner (DPA) settings by case criteria from the Treatment Planning Software 307. From this combination of received information, the prescription service (RX Service) module 317 generates an updated product setting file 318, which includes the latest/updated passive aligner (DPA) settings. There is no need to update the Treatment Planning Software 307 configuration to get the latest passive aligner (DPA) settings.

The Treatment Planning Software 307 uses the latest passive aligner (DPA) settings to generate one or more treatment plans and saves the latest passive aligner (DPA) settings in Treatment Case Files (ADF) 309, which can include other aspects of the treatment plans. The latest passive aligner (DPA) settings may be transferred to all applications where they are needed via the Treatment Case Files (ADF) 309. For example, the passive aligner (DPA) settings may be presented to a corresponding doctor via a client-based treatment planning software application (ClinCheck) 311, which is part of the treatment management module 121. The client-based treatment planning software application may be configured to allow a doctor to modifying one or more aspects of a treatment plan generated by the treatment planning software 307. After making such modifications, the doctor may submit these changes so that the treatment planning software 307 can generate one or more new treatment plans.

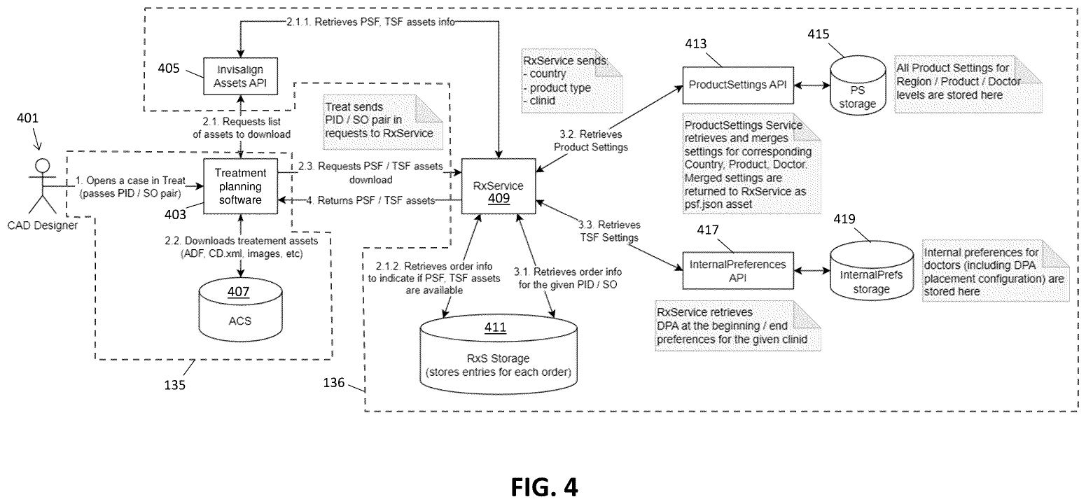

FIG. 4 shows a diagram indicating an example of an order opening process showing the usage of product setting files in treatment planning software. A CAD designer 401 opens a case in the Treatment Planning Software 403 (Action 1.), where the case is verified by passing PID/SO pair. The Treatment Planning Software 403 requests a list of assets to download from an Assets application programing interface (API) 405 (Action 2.1.). The Assets API 405 retrieves product setting file (PSF) and treatment setting file (TSF) assets information from a prescription service (RX Service) module 409 (Action 2.1.1.). The prescription service (RX Service) module 409 retrieves order information, which indicates whether the product setting file (PSF) and treatment setting file (TSF) assets are available, from a prescription storage database 411 (Action 2.1.2.). The prescription storage (RxS) database 411 is structured to store entries for PSF and TSF assets for each order and treatment type. The Treatment Planning Software 403 also downloads treatment assets (ADF, CD.xml, images, etc.) from an ACS database 407 (Action 2.2.). Treatment Planning Software 403 then requests/downloads PSF/TSF assets from the prescription service (RX Service) module 409 (Action 2.3.). The Treatment Planning Software 403 sends PID/SO pair in requests to the prescription service (RX Service) module 409.

The prescription service (RX Service) module 409 retrieves order information for the given PID/SO from the prescription storage (RxS) database 411 (Action 3.1.). The prescription service (RX Service) module 409 also sends the jurisdiction (e.g., country), product type, and doctor identification (clinID) to a Product Settings Service API 413 to retrieve Product Settings (e.g., 209) (Action 3.2.). The Product Settings Service API 413 retrieves and merges settings for corresponding jurisdiction (e.g., country), product type, and doctor identification from a Product Setting (PS) Storage database 415. The Product Setting (PS) Storage database 415 stores all product setting by jurisdiction (e.g., country or region), product type, and doctor. The merged settings are then returned to the prescription service (RX Service) module 409 in a Product Settings File (e.g., 207) that is in a text format.

The prescription service (RX Service) module 409 also retrieves treatment setting file (TSF) assets from an Internal Preferences API 417 (Action 3.3.). The prescription service (RX Service) module 409 may retrieve the passive aligner (DPA) settings at the beginning and/or end of the preferences for the given doctor (clinID). The Internal Preferences API 417 retrieves internal preferences for doctors (including passive aligner (DPA) placement configurations) from an Internal Preferences Storage database 419.

Once the prescription service (RX Service) module 409 has retrieved the product settings (PS) from the Product Settings Service API 413 and the treatment settings (TS) from Internal Preferences API 417, the prescription service (RX Service) module 409 can store this information in the prescription storage (RxS) database 411 and return the product setting file (PSF) and treatment setting file (TSF) assets to the Treatment Planning Software 403 (Action 4.). The product setting file (PSF) and the treatment setting file (TSF) may be text files, for example, that may be in a human-readable format.

Thus, when the CAD designer 401 opens an order in the Treatment Planning Software 403, the Treatment Planning Software 403 downloads the product setting file (PSF) and treatment setting file (TSF) from the prescription service (RX Service) module 409 and applies these settings to the current treatment case. The CAD designer 401 may then use the Treatment Planning Software 403 to generate one or more treatment plans based on the settings from the product setting file (PSF) and treatment setting file (TSF). That is, one or more of the treatment plans may include one or more passive aligners (DPA) based on the parameters of the product setting file (PSF) and treatment setting file (TSF).

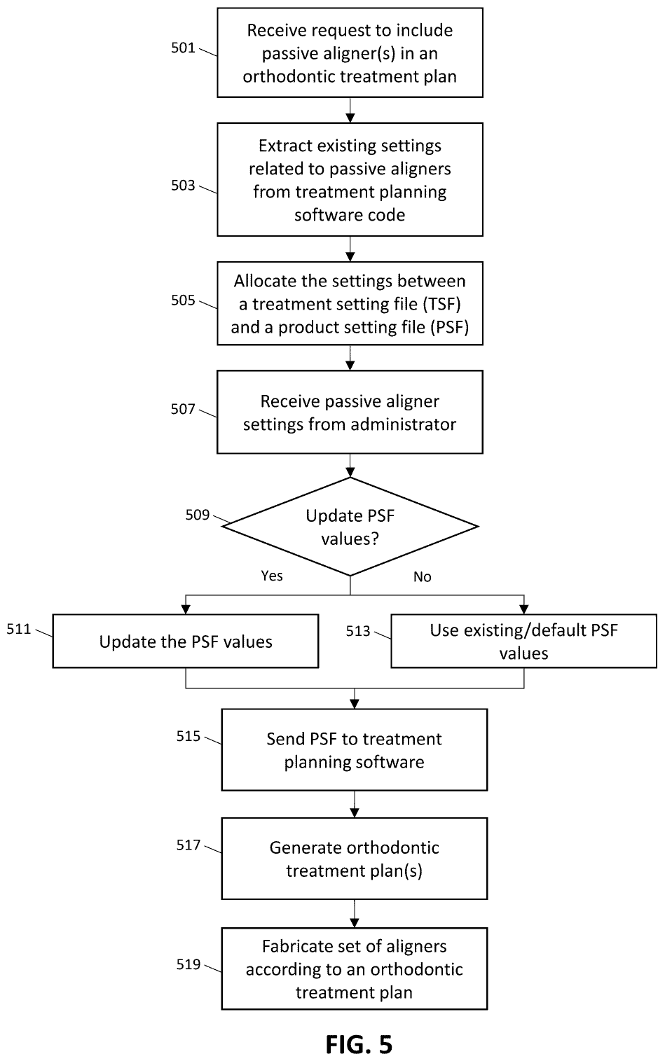

FIG. 5 is a flowchart indicating an example process for generating an orthodontic treatment plan and fabricating a set of aligners that include one or more passive aligners (e.g., dual passive aligners (DPA)). At 501, a request to include one or more passive aligners in the orthodontic treatment plan is received. The request may be received from a doctor and/or a treatment plan designer (e.g., CAD designer).

At 503, existing (e.g., default) settings related to passive aligners are extracted from treatment planning software code of a treatment planning software. The settings may include values based to jurisdiction (e.g., country or region), product (e.g., type of aligners), doctor identification (e.g., doctor's permission to use passive aligners), placement of passive aligners (e.g., beginning, middle or end of treatment plan), and/or other factors. The settings may be extracted by a prescription service (RX Service) module, which may be a cloud-based service that is separate from the treatment planning software module.

At 505, the prescription service (RX Service) module creates a treatment setting file (TSF) and a product setting file (PSF) and allocates the settings from the treatment planning software code between the treatment setting file (TSF) and the product setting file (PSF). For example, settings specific to the particular product (e.g., jurisdiction (e.g., country or region), product type (e.g., type of aligners), and doctor identification (e.g., doctor's permission to use passive aligners)) may be allocated in the product setting file (PSF), and settings specific to the treatment plan (e.g., placement of passive aligners (e.g., beginning, middle or end of treatment plan)) may be allocated in the treatment setting file (TSF). The treatment settings file and the product settings file have different updating privileges. For example, the product setting file (PSF) may contain settings that can be changed/updated during the treatment process (e.g., by a technician). The treatment setting file (TSF) may contain settings that are specific to the current product and that are unchangeable at the prescription service (RX Service) module (e.g., by the technician). The PSF and TSF files may be saved in a prescription storage (RxS) database 411 that is structured to store entries for PSF and TSF assets (values) for each order and treatment type. For example, the PSF may include values associated with jurisdiction (e.g., country or region), product type (e.g., type of aligners), and doctor identification (e.g., doctor identification number and/or doctor's permission to use passive aligners). The TSF may include values associated with internal preferences associated with each doctor (e.g., placement of passive aligners (e.g., beginning, middle or end of treatment plan)).

At 507, the prescription service (RX Service) receives passive aligner settings from a product settings administrator. The product settings administrator may define passive aligner settings based on one or more criteria. For instance, passive aligners may be allowed in some jurisdictions (e.g., countries or regions) and not allowed in other jurisdictions. In addition, the maximum number of passive aligners may differ from jurisdiction to jurisdiction. Whether passive aligners are allowed, and the maximum number of allowed passive aligners (treatment stages with passive aligners), may also depend on the particular doctor (e.g., may vary from doctor to doctor). treatment type (e.g., some treatment types may not allow for passive aligners), and/or case flow.

At 509, the prescription service (RX Service) module determines whether the PSF values are to be updated. The prescription service (RX Service) module can compare the passive aligner settings received from a product settings administrator with the existing PSF values (e.g., corresponding to the extracted values from the treatment planning software code). If these values differ, the PSF values may be updated according to the passive aligner settings received from a product settings administrator. If these values are the same, the PSF values do not need updating.

At 511, the PSF values are updated according to the passive aligner settings received from a product settings administrator. For example, typically the default product settings (205) do not include passive aligners. Thus, if the prescription service (RX Service) module determines that passive aligners are allowed, the PSF values may be updated to include passive aligners and the maximum number of passive aligners according to the passive aligner settings received from a product settings administrator.

At 513, the PSF values are not updated and the existing (e.g., default) PSF values are used. For example, if existing/default values extracted from the treatment planning software do not include passive aligners, the PSF values will also not include passive aligners.

At 515, the PSF (updated or not updated) is sent to the treatment planning software. PSF values associated with passive aligners may be incorporated into the treatment planning software. Since only certain aspects related to passive aligners are in the PSF, only these values are incorporated into the code. For example, passive aligner values allocated to the treatment setting file (TSF) may remain unchanged. This allows certain values associated with passive aligners to be changed (via the prescription service (RX Service) module) while leaving other values associated with passive aligners to remain unchanged.

At 517, the treatment planning software generates one or more orthodontic treatment plans. If the PSF was updated to include passive aligners, the one or more orthodontic treatment plans may include one or more stages that includes passive aligners. The one or more orthodontic treatment plans may also consider the maximum number of allowed passive aligners as dictated by the PSF. If the PSF was not updated and does not include passive aligners, the one or more orthodontic treatment plans may not include one or more stages that includes passive aligners.

At 519, a set of aligners is fabricated according to an orthodontic treatment plan. For example, the doctor may choose a particular orthodontic treatment plan from multiple orthodontic treatment plans generated by the treatment planning software and presented to the doctor. The selected orthodontic treatment plan may include one or more stages that includes a passive aligner. Instructions for fabricating the set of aligners based on the selected orthodontic treatment plan may be sent to one or more manufacturing apparatuses to fabricate the set of aligners.

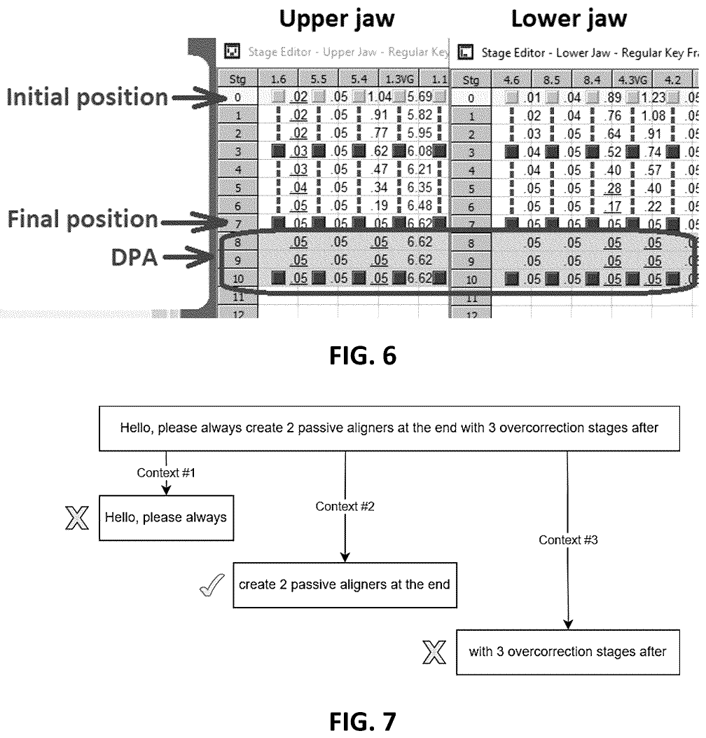

FIG. 6 shows an example user interface illustrating an example chart showing tooth planned movements for the upper and lower jaws at different stages of a treatment plan. In this example, three stages of the treatment plan include dual passive aligners (DPA). In this case, the DPAs are added after a final teeth positioning stage.

Described herein are different ways in which dental practitioners can request passive aligners during the building of a treatment plan. As discussed, the computing environment (e.g., doctor system 120) may be configured to provide a user interface for a dental practitioner to enter a request for a passive aligner and request a certain number of passive aligners. In some cases, the user interface includes an area (e.g., comments section) that allows for entry of the request via a free form written request. A CAD designer may then receive the written request and incorporate the passive aligner(s) into a precalculated treatment plan without passive aligner(s) calculated by the treatment planning software. For example, a work instruction for the CAD designer may be created (e.g., after stages are built) to check whether the dental practitioner made a passive aligner request. If one or more passive aligners are requested, the CAD Designer may use a corresponding tool provided by the treatment planning software to specify the number and position of requested passive aligner(s).

In some cases, free from written requests for passive aligners may lead to miscommunication between dental practitioner and the CAD designer, which may lead to delays and/or additional costs. For example, dental practitioners from different countries, cultures, and orthodontics traditions, may result in a variety of wordings for the passive aligner requests.

To solve this problem, the software (e.g., of doctor system 120) may include a passive aligner request interpreting module (e.g., 126 of FIG. 1) that is configured to interpret requests (e.g., from a dental practitioner) for including one or more passive aligners in the treatment plan. The passive aligner request interpreting module may be configured to extract information related to passive aligner requests from free-form comments (e.g., from the dental practitioner) by filtering out non-related information (e.g., words) from the comments. Natural language recognition and natural language processing (NLP) may be used to read written requests. The passive aligner request interpreting module may include any of a number of different types of analytical techniques, such as symbolic-based language processing, statistical-based language processing and/or neural network-based language processing. The passive aligner request interpreting module may be configured to parse free-form comments (e.g., from a dental practitioner) using neural network technology and transform free-form comments to formal instructions used during automated treatment planning by the treatment planning software (e.g., 135 of FIG. 1).

The passive aligner request interpreting module provides automation of passive aligner placement that otherwise would require a CAD designer manual setup. This can reduce errors caused by human factors related to understanding of passive aligner request. It can also provide time costs reduction for passive aligner placement setup.

In some examples, the neural network model includes a long short-term memory (LSTM) recurrent neural network (RNN). A RNN is a type of neural network with a recurrent architecture. The RNN may be configured to store an internal state, which allows for the analysis of a whole sentence instead of separate words of a sentence. LSTM is a further expansion of RNN architecture that allows for the understanding of the context of processed words. For example, the LSTM may be configured to determine whether the words are still actual and relate to the current context, or whether the words relate to a previous part of a sentence. The LSTM RNN may be configured to match different words in a sentence with a single passive aligner placement request, ignoring other requests, even if the other requests are in the same sentence. This may be useful when there are other numbers near “passive aligners” words that relate to different context.

FIG. 7 illustrates an example of context interpretation using an example LSTM RNN. A free form request includes the phrase “Hello, please always create 2 passive aligners at the end with 3 overcorrection stages after”. The LSTM RNN may be configured to process the groups of words “Hello, please always”, “create 2 passive aligners at the end”, and “with 3 overcorrection stages after” using different contexts. In this case, the LSTM RNN used the group of words “create 2 passive aligners at the end” to integrate the passive aligner request, while ignoring the groups of words “Hello, please always” and “with 3 overcorrection stages after”.

The system may be configured to use an NPL (e.g., LSTM RNN) to interpret free form comments (e.g., for every case), convert the comments into formal instructions that are understandable by treatment planning software, and provide the formal instructions as an input for the further treatment planning processing.

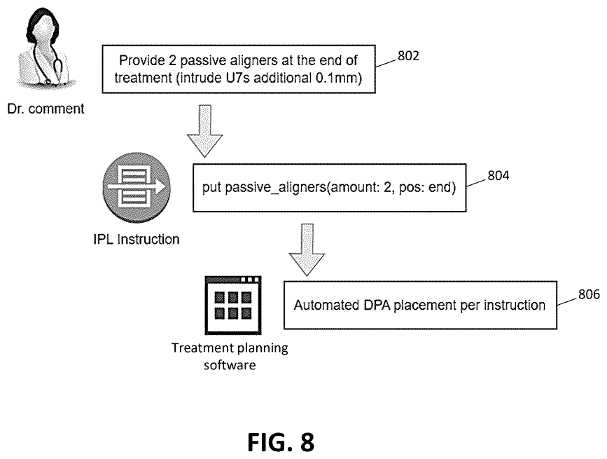

FIG. 8 shows an example workflow for including a passive aligner in a treatment plan. At step 802, a dental practitioner requests one or more passive aligners via comments in a user interface (e.g., of a prescription service module 136). The request may be in plain text. The number of sentences that may be (but not necessarily) separated with newlines or dots. In some examples, the system includes a neural network (e.g., LSTM RNN) architecture that works with sentences, with each separate sentence used as an input to the neural network. The output of each provided sentence may correspond to the number of passive aligners that is requested to be placed. In some cases, the number of passive aligners may be zero (0) if no passive aligners are requested by the given input sentence.

At step 804, a protocol language (e.g., Invisalign Protocol Language (IPL)) script converts the output into instructions with corresponding parameters. The protocol language instruction may be in a format that is readable as input by the treatment planning software. The protocol language instruction may be stored in the computing environment along with other clinical protocols.

At step 806, the treatment planning software reads the protocol language instructions. The treatment planning software may request all the required assets for the given case. Among all other assets, treatment planning software may call the service that is responsible for storing clinical protocols and request all available protocols for the given case. As a response the treatment planning software may acquire a newly created protocol with a passive aligner placement request and import it to the treatment plan. After the required protocol(s) is/are imported, the treatment planning software may automatically build a treatment plan in accordance with all imported protocols and add the required number of passive aligners to the treatment plan. The information regarding the passive aligner is stored in the case as a part of the treatment plan. This means that the CAD designer does not have to place the passive aligner(s) manually in future revisions.

The lifecycle of neural networks may include two main phases: training and prediction. Prediction mode is used in production and produces the result based on the provided input. However, every network may be trained beforehand to be able to produce the desired result. In some examples, a classic state-of-art backpropagation learning algorithm is used, which is well suited for training multilayered neural networks using datasets with ground truth (or with markup). Datasets with ground truth (or with markup) include a list of pairs input and output.

Generally, a “good” dataset for neural networks may include one or more of the following characteristics: 1) cover the whole scope of the problem that neural network is meant to solve; 2) provide a data quality-dataset is trustworthy and clinically valid; 3) have an adequate size-too few examples lead to an undertrained neural network that will not be able to predict the desired result even on simple inputs, while providing too many examples leads to overtrained neural network that will not be able to generalize the input and will provide a good prediction only for inputs that it was trained on; 4) have no bias-biased dataset results in the biased neural network; and 5) be diverse-simply copying the same input a thousand times will not make it better but may make it even worse. The richer the experience of neural network during the training, the better generalized rules it will have during the prediction phase.

In one example, a clinical database characterized as having all the “good” qualities mentioned above contained billions of dental practitioner comments collected over decades. For simplicity reasons for a prototype only English comments were selected, but the approach works similarly with any other language and does not depend on any particular language. Moreover, the neural network may not work directly with words, but with a word's representation instead. For training purposes, 100,000 comments were selected randomly during the last two years from a production environment database. Thus, the neural network may be trained using numerous comments for numerous different patient cases.

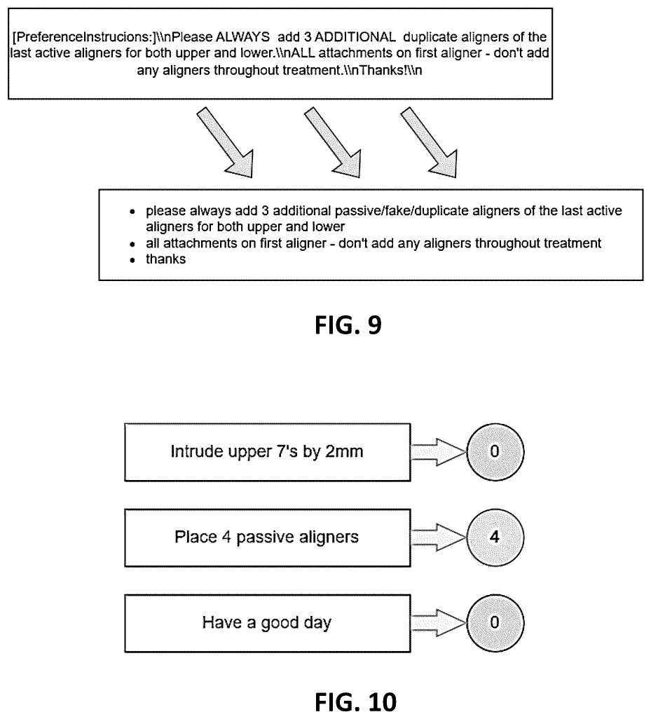

The format of the data stored in the clinical database may not fit specific needs, so an automated dataset preparation script may be performed. FIG. 9 shows a diagram illustrating an example use of an automated dataset preparation script. The automated dataset preparation script may split comments into groups (e.g., of words, sentences, or phrases), using semicolons (;) and newline characters (\\n) as separators, remove special markup symbols and tags, unprintable symbols, trailing and duplicated spaces, and other transforms that do not change the semantic of the comment. A dataset of the mentioned size may fit needs of a prototype solution, but a dataset for a production-ready neural network may be determined based on supervising the training process, which may be a non-trivial task that involves monitoring of training metrics. Other dataset parameters may be estimated after preparing the dataset ground truth.

FIG. 10 shows a diagram illustrating an example classification using a dataset markup (or ground truth). The dataset markup (or ground truth) suggests that every training input has a corresponding desired output. This input/output pair may be used in a backpropagation algorithm that teaches internal neural network the parameters to produce the desired output for the given input. The problem to solve is a “multiclass classification” problem. That is, for an arbitrary input, the neural network can provide a “class” that the input belongs to. For example, integer classes may be introduced, where a number represents the actual number of passive aligners requested for placement. For instance, Class zero (0) may correspond to no requested passive aligners, while class four (4) may correspond to four (4) requested passive aligners.

The dataset markup tool may assign a corresponding class for each training input (sentence in this case). It may take about five seconds for a human to read, understand, and assign the correct output class for a single input. Multiplying this time by the size of the dataset, such work is estimated to take about 140 hours to complete. For the best markup quality, it may be beneficial to split the work among several clinically experienced persons and perform the work manually. However, for prototype purposes an ad-hoc semi-automated markup tool (e.g., python script) may be implemented, which uses regular expressions to look for a known pattern of requesting passive aligners. If the dataset markup tool determines that a given comment docs not have anything about passive aligners, the class may automatically be marked as zero (0). If the dataset markup tool determines that the given comment includes a request for at least one passive aligner, the class may automatically be marked according to the corresponding class (e.g., 1, 2, 3, etc.). If the dataset markup tool does provide a clear class determination (e.g., not 100% sure), the markup may remain empty, leaving it to manual parsing by a human. Rules for the dataset markup tool may be heuristic. For example, they may include checks for line length, looking for candidate words (“passive”, “copy”, “fake”, “dpa”, . . . ), stop words (“fake ipr”), numbers in the sentence (“four”, “1”, . . . ), and/or candidate words, numbers, or symbols.

In one example, the dataset markup tool was used to automatically markup about 80% of a large dataset, with the remaining processed manually. For production purposes, the dataset ground truth may be validated with the clinical team because it may impact the clinical outcome of the treatment plan. For prototype purposes such validation may be omitted.

After the dataset markup is ready, it may be validated on the quality, diversity, and other required parameters. All desired classes should be presented in the dataset in sufficient quantity. Based on the statistics of classes met in the dataset, it may be expanded and/or reduced. Usually the decision is taken in conjunction with the results of network training supervision results.

The neural network may be trained using any of a number of neural network techniques. For example, a backpropagation algorithm may be used. In some cases, the backpropagation algorithm uses the Adam optimization. The backpropagation algorithm may require modifications to be trained using the passive aligner related dataset. Firstly, every input line may be tokenized (split into separate words). Given that the neural network does not work with words—it works with numbers—an identifier is assigned for every word (an integer value) and every word is replaced with the corresponding value. This map of words and identifiers is called “dictionary”, which is stored and reused during the prediction phase. Then every input line is padded to the same length for optimization reasons of the training algorithm. The padding length is chosen as the maximum of all input lengths. Lines that are shorter are filled at the end with fake words (e.g., zeros).

At this moment the neural network still has no idea about the words semantic. For instance, the words “passive aligners” and “fake aligners” are different words for the neural network. However they are almost synonymous in the context of the problem. To capture the semantics of the words an embedding layer is introduced to the architecture of the neural network. The aim of the embedding layer is to transform every word (an integer value at this point) to a semantic-meaning space where words such as “passive” and “fake” are close to each other while words such as “passive” and “non-passive” are far from each other even if they are close to each other lexicographically.

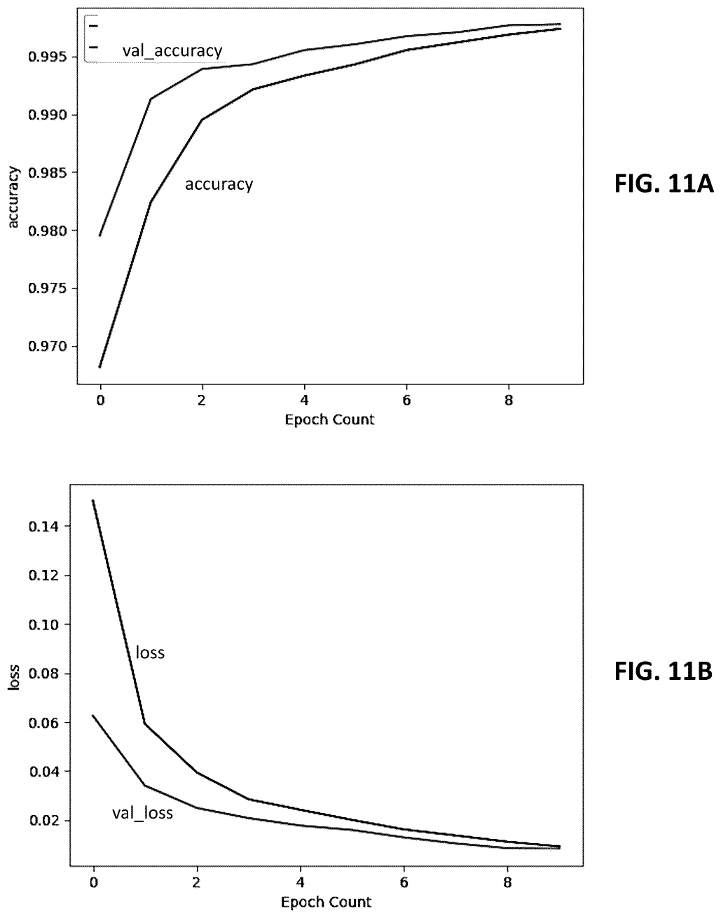

FIGS. 11A and 11B show example epoch counts of an example prototype training. Training parameters for the prototype are chosen heuristically during training supervision. Exact parameters and values can be found in the source code. Training is supervised by monitoring convergence of training metrics “accuracy”, “validation accuracy” (val_accuracy), “loss”, and “validation loss” (val_loss). Training time varies from a few hours to tens of hours and mostly depends on the dataset volume and desired model quality. The prototype training took about an hour on the regular developer machine CPU. Production ready state training is estimated to take about 10-20 hours. It is a one-time operation that does not require a high-performance machine.

After neural network is trained, it is validated. For validations purposes a dataset with markup that neural network has never seen before is used for the sake of experiment purity. The original dataset is split into two parts-training dataset (80%) and validation dataset (20%). The training dataset is used only in the training process, while the validation dataset is used only during validation process. Table 1 shows the correct class detection success rate that was achieved for the prototype.

TABLE 1

| | Correct class detection success rate |
|---|---|
| DPA are requested | ~97% |
| DPA not requested | ~99.9% |

The “DPA are requested” is considered a success if the class is correctly predicted (actual number of requested DPAs), not simply that some number of DPAs were requested. Type I errors (false positive) in this case are situations when the neural network detects that a DPA is requested, though input string belongs to class zero (0). Type II errors (false negative) in this case are situations when the neural network detects that no DPAs are requested, however input string contains a request to place one or more DPAs. These two types of errors have different risks. False positive errors lead to DPA placement while the dental practitioner doesn't request it—it may confuse the CAD designer and lead to additional time costs. However, false negative errors have lesser risks and simply lead to the existing behavior where the CAD designer places the DPA(s) manually. These risks can be balanced by manipulating the input dataset markup. Contradictory input that is difficult to parse can be assigned to class zero (0) to leave the decision on CAD designer. This can reduce type I errors with a bigger risk, while increase type II errors with a much lesser risk.

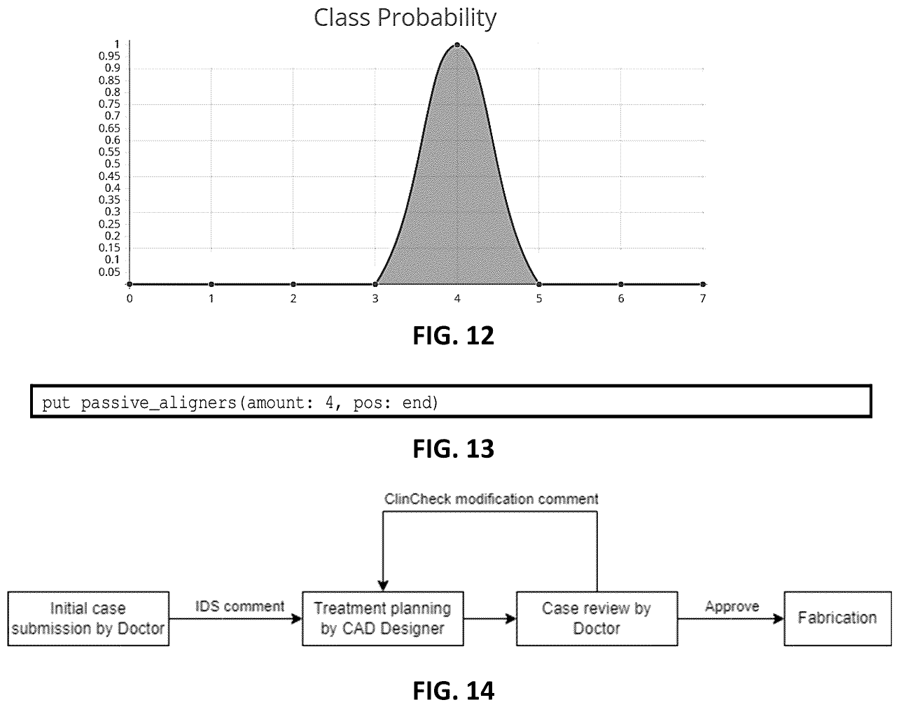

At this point neural network is ready for usage in prediction mode. For the best results, any input given to the neural network follows the same operations as the dataset. It is split by the “newline” and semicolon symbols into separate lines, invalid characters are removed, and so on. Then, the input line is tokenized, converted to integer values, and padded using the same dictionary and padding settings that were used during the model training process. The output of neural network model is a probability distribution, which indicates how certain the neural network is that the input string belongs to one of the classes. FIG. 12 shows a probability distribution of an example neural network output for an input string that requests four DPAs. To determine the neural network prediction class, the maximum probability may be found among the distribution. For this example, it is class 4. The prediction algorithm of the prototype works for less than a second (e.g., about 0.1 sec for a warm start when the script, dictionary, and the model itself are already loaded to the memory).

The protocol language (IPL) instruction is a human-readable instruction that formally describes some clinical aspects of the treatment protocol. However, the treatment planning software works with Treatment Settings Format (TSF) settings. These two entities (TSF setting and IPL instruction) are mapped to each other. In the production flow, the IPL instructions are acquired from protocols collection and are converted into TSF settings using an IPL compiler. Therefore, the IPL instruction can be generated and converted to TSF later at some point.

FIG. 13 shows an example IPL instruction for passive aligner placement. In this example, the IPL instructions specify 4 passive aligners at the end of the treatment plan. However, the IPL instructions may specify any number of passive aligners (e.g., 1, 2, 3, 4, 5, or more) and any of a number of placements (e.g., beginning, middle or end) of the treatment plan depending on the detected class and placement. If neural network detects a zero class (no passive aligners are required), there is no need to generate an instruction at all. If neural network detects any other class, this class is put to the IPL instruction template as a value of “amount” parameter. The method of storage and delivery of IPL instruction to the treatment planning software may vary. However, existing services like ProtocolsAPI and RxService can provide this option.

To achieve automated passive aligner placement in the treatment plan, the treatment planning software may download all assets that belong to the current case, including any newly generated instruction about passive aligner placement as a separate protocol. All protocols associated with the case are automatically imported to the case, which means that all imported TSF settings will be considered during the automated treatment preparation. Passive aligners may automatically placed right after staging creation in accordance with the corresponding protocol that was generated by the neural network. All features that will be placed afterwards comply with the passive aligner feature and require no specific action from the CAD designer. The passive aligner information (e.g., number of passive aligners) is stored in the case file along with the placement instruction. This means that all downstream applications will receive a valid treatment plan with a requested number of passive aligners. Even in the case where staging or passive aligner(s) are to be rebuilt, the software can automatically place the passive aligner(s) the same way as for the initial treatment plan.

This automated passive aligner placement saves several seconds for a CAD designer that they spend on: opening prescription and manually parsing dental practitioner comments in search of passive aligner instructions (e.g., several seconds); opening the passive aligner placement tool, configuring passive aligner setup, and waiting for placement of the passive aligner(s) (e.g., a few more seconds). Time savings may not be big (about 5-10 seconds) for a single case. However, significant time savings may be achieved when considering a large number of cases.

There are a number of different options for incorporating automated passive aligner placement into treatment planning workflow. In some production workflows, there are two primary ways of delivering dental practitioner comments to a CAD designer: 1) in the service management module (e.g., 124 in FIG. 1 (e.g., IDS)) during the initial case submission, and 2) in the treatment management module (e.g., 121 in FIG. 1 (e.g., ClinCheck)) during case review. FIG. 14 shows an example workflow. The automated passive aligner placement solves the problem of parsing comments from the service management module (e.g., IDS) during the initial case submission. The same approach can be used for parsing comments from the treatment management module (e.g., ClinCheck) when the dental practitioner decides to modify the treatment plan or notices an error made by the CAD designer. Thus, it provides the possibility of not only fixing an error automatically, but also to retrain the neural network to avoid such errors in future.

Model quality depends on the dataset and markup that it is trained on. By default, a neural network does not know that words “four” and “4” correspond to the same number. It is a general problem of semantic understanding. In the same way, the words “passive” and “not-active” may have nothing in common for the neural network. A dataset can explicitly have these examples marked with the same output class so that the neural network can establish these relationships.

Model instability due to typos or paraphrases in the input text may be addressed by explicitly added them to the training dataset. For example, the words “psasive” and “aligner” may be unknown to the neural network unless they are explicitly added to the training dataset. The same can be done for complicated wording or paraphrasing. For example, the dental practitioner may use “I want the smallest minimal movement (or total absence of it) on the lower jaw” instead of “passive aligners”. Similar additions to the training dataset may include those for correctly parsing the text “Create passive aligners at stages 6-8” such that the result is class “3” instead of class “6”. Conditional statements may be handled by the protocol language (e.g., IPL).

Another problem is the limitation of input parameters that the current model can predict. For example, correct parsing of input “4 passives at the start of the upper jaw” requires introduction of multidimensional output-several classes for amount of DPA, at least three classes for its position (at start, at end, at both start and end), and at least three classes for jaws (upper, lower, both). Workable solutions for these situations are to either increase dataset (exponentially for adding every new parameter) or use separate models for detecting separate parameters.

A workable solution for the problems mentioned above may include using a pretrained neural network that already knows how to understand the word semantic and can fix typos. This may be achieved, for example, using artificial intelligence, such as a large language model (e.g., ChatGPT 3.5 and ChatGPT 4 models). Among other advantages these models also do not require as large of a dataset. Providing just a few examples only for a few classes may be enough for a large language model to generalize the problem and provide good decisions for the whole problem.

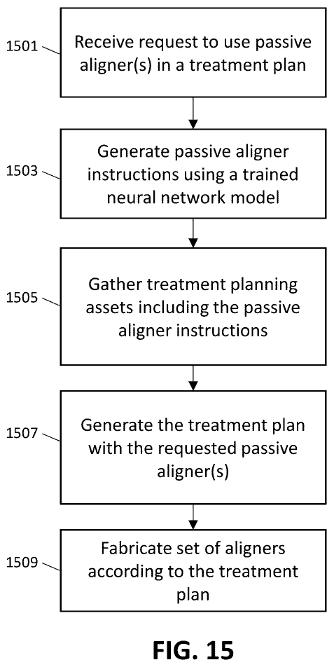

FIG. 15 shows a flowchart indicating an example process for generating a treatment plan that includes using a neural network to interpret requests for passive aligners. At 1501, a request to include one or more passive aligners in the orthodontic treatment plan is received. The request may be expressed in free form (e.g., natural language or text form). For example, the request may be in written text form in a comment section entered by a user (e.g., dental practitioner) via a user interface. The request may originate from a remote computer, such as at an office of a doctor or dental practitioner.

At 1503, passive aligner instructions are generated using a trained neural network model. The trained neural network generates the passive aligner instructions based on interpreting the request to include the one or more passive aligner. In some examples, this involves splitting the request into groups of words, sentences or phrases and separating the groups of words, sentences or phrases with separators, such as semicolons and/or newline characters. In some examples, the neural network model is trained using a backpropagation learning algorithm. In some examples, the neural network model is a recurrent neural network model, such as a long short-term memory (LSTM) recurrent neural network model. The trained neural network model may be trained based on a large number of requests (e.g., about 100,000 requests or greater) associated with different patient cases. The neural network model may be configured to tokenize the request. For example, the neural network model may be configured to assign an identifier for each word, sentence, or phrase of the request. The passive aligner instructions include information related to the request, such as a number of requested passive aligners and a placement of the requested passive aligners in the orthodontic treatment plan (e.g., beginning, middle, or end). In some cases, an embedding layer is used to transform words (e.g., symbols, phrases, etc.) of the request to semantic-meaning space. In some examples, generating the passive aligner instructions includes converting the instructions to a format that is readable by the treatment planning software module. For example, the instructions may be converted from a protocol language (e.g., IPL) to a treatment settings file (e.g., TSF) format.

At 1505, treatment planning assets, including the passive aligner instructions, are gathered. The treatment planning assets may include information needed for generating the treatment plan. The treatment planning assets may be gathered from one or more (e.g., remote) computers using one or more computer services/modules. This information may be gathered by the treatment planning module.

At 1507, the treatment planning module generates the treatment plan with the requested one or more passive aligners. Generating the orthodontic treatment plan can include determining which one or more stages of the treatment plan include the requested one or more passive aligners. The requested one or more passive aligners can be integrated in corresponding one or more stages of the treatment plan after the stages are created.

At 1509, a set of aligners is fabricated according to the treatment plan. The set of aligners includes one or more passive aligners based on the attributes of the request (e.g., from the dental practitioner).

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for case of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method, the method comprising:
receiving a request to include passive aligners in an orthodontic treatment plan;
extracting settings related to passive aligners from treatment planning software code of a treatment planning software module;
accessing a treatment settings file and a product settings file, wherein the extracted settings are allocated between the treatment settings file and the product settings file, the treatment settings file including settings specific to the orthodontic treatment plan, and the product settings file including settings related to a particular product, wherein the treatment settings file and the product settings file have different updating privileges;
updating the product settings file based on passive aligner settings received from a product settings administrator, wherein the product settings file is updated to indicate inclusion of passive aligners and a maximum number of allowed passive aligners;
sending the updated product settings file to the treatment planning software module, wherein the treatment planning software module updates the treatment planning software code to include the passive aligners and the maximum number of allowed passive aligners according to the updated product settings file; and
generating the orthodontic treatment plan via the treatment planning software module, wherein the orthodontic treatment plan includes one or more stages that includes passive aligners, and wherein a number of the one or more stages is not greater than the maximum number of allowed passive aligners.

2. The method of claim 1, further comprising applying orthodontic treatment plan to treat the patient.

3. The method of claim 1, further comprising creating the treatment settings file and the product settings file and allocating the extracted settings between the treatment settings file and the product settings file.

4. The method of claim 1, wherein the settings related to passive aligners are extracted from the treatment planning software code of the treatment planning software module by a prescription service module.

5. The method of claim 4, wherein the prescription service module is a cloud-based service module.

6. The method of claim 4, wherein the prescription service module is separate from the treatment planning software module.

7. The method of claim 4, wherein the settings of the treatment settings file are unchangeable via the prescription service module.

8. The method of claim 4, wherein the prescription service module saves the updated product settings file in a cloud-based prescription storage database.

9. The method of claim 1, wherein extracting the settings related to passive aligners comprises extracting the settings specific to the orthodontic treatment plan via an internal preferences application programing interface (API), and extracting the settings related to the particular product via a product settings API.

10. The method of claim 1, further comprising opening a case file in the treatment planning software module in response to receiving the request to include the passive aligners.

11. The method of claim 10, wherein a CAD designer opens the case file.

12. The method of claim 1, wherein the request to include the passive aligners is received from a doctor.

13. The method of claim 1, further comprising presenting an indication of allowance of passive aligners and the maximum number of allowed passive aligners according to the updated product settings file to a doctor via a client-based treatment planning software module.

14. The method of claim 1, further comprising presenting the orthodontic treatment plan to a dental practitioner via a display.

15. The method of claim 1, further comprising sending instructions to one or more manufacturing apparatuses to fabricate a set of aligners with one or more passive aligners based on the orthodontic treatment plan.

16. A system comprising:
one or more processors; and
one or more memory stores coupled to the one or more processors, wherein the one or more memory stores store computer-program instructions that, when executed by the one or more processors, perform a computer-implemented method comprising:
receiving a request to include passive aligners in an orthodontic treatment plan;
extracting settings related to passive aligners from treatment planning software code of a treatment planning software module;
creating a treatment settings file and a product settings file and allocating the extracted settings between the treatment settings file and the product settings file, the treatment settings file including settings specific to the orthodontic treatment plan, and the product settings file including settings related to a particular product, wherein the treatment settings file and the product settings file have different updating privileges;
updating the product settings file based on passive aligner settings received from a product settings administrator, wherein the product settings file is updated to indicate inclusion of passive aligners and a maximum number of allowed passive aligners;
sending the updated product settings file to the treatment planning software module, wherein the treatment planning software module updates the treatment planning software code to include the passive aligners and the maximum number of allowed passive aligners according to the updated product settings file; and
generating the orthodontic treatment plan via the treatment planning software module, wherein the orthodontic treatment plan includes one or more stages that includes passive aligners, and wherein a number of the one or more stages is not greater than the maximum number of allowed passive aligners.

17. The system of claim 16, wherein the computer-implemented method further comprises sending instructions to one or more manufacturing apparatuses to fabricate a set of aligners with one or more passive aligners based on the orthodontic treatment plan.

18. The system of claim 16, wherein the settings related to the particular product include jurisdiction, product type, and doctor identification.

19. The system of claim 16, wherein the settings specific to the orthodontic treatment plan include placement configuration of the passive aligners in the orthodontic treatment plan.

20.-25. (canceled)

26. A non-transitory computing device readable medium having instructions stored thereon that are executable by one or more processors to cause one or more computing devices to perform a method comprising:
receiving a request to include passive aligners in an orthodontic treatment plan;
extracting settings related to passive aligners from treatment planning software code of a treatment planning software module;
creating a treatment settings file and a product settings file and allocating the extracted settings between the treatment settings file and the product settings file, the treatment settings file including settings specific to the orthodontic treatment plan, and the product settings file including settings related to a particular product, wherein the treatment settings file and the product settings file have different updating privileges;
updating the product settings file based on passive aligner settings received from a product settings administrator, wherein the product settings file is updated to indicate inclusion of passive aligners and a maximum number of allowed passive aligners;
sending the updated product settings file to the treatment planning software module, wherein the treatment planning software module updates the treatment planning software code to include the passive aligners and the maximum number of allowed passive aligners according to the updated product settings file; and
generating the orthodontic treatment plan via the treatment planning software module, wherein the orthodontic treatment plan includes one or more stages that includes passive aligners, and wherein a number of the one or more stages is not greater than the maximum number of allowed passive aligners.

27.-68. (canceled)
